(19) United States
(12) Patent Application Publication
(10) Pub. No.: US 2014/0161750 A1
(43) Pub. Date: Jun. 12, 2014

(54) SUNSCREEN COMPOSITION WITH PIGMENT CORRECTING PROPERTIES

(71) Applicant: Thomas Christopher Balshi, Delray Beach, FL (US)

(72) Inventor: Thomas Christopher Balshi, Delray Beach, FL (US)

(21) Appl. No.: 13/712,594

(22) Filed: Dec. 12, 2012

(51) Int. Cl. A61K 8/97; A61Q 17/04; A61K 8/49; A61Q 19/00; A61K 8/37; A61K 8/60

(52) U.S. Cl. CPC A61K 8/97; A61K 8/37; A61K 8/602; A61K 8/4973; A61K 8/4926; A61Q 19/00; A61Q 17/04
USPC 424/60

(57) ABSTRACT

The present invention relates to a sunscreen composition that provides for pigment correction comprising: zinc oxide; octinoxate; α-arbutin; ascorbic acid; glycyrrhiza glabra extract; green tea extract; pomegranate extract; cucumber extract; niacinamide; and a carrier. The composition can be used to treat a variety of pigment issues including lentigo, liver spots, melasma, post-inflammatory pigment issues, sun spots, freckles, and age spots.

### FIELD OF THE INVENTION

The present invention relates to novel compositions comprising a sunscreen that provides for pigment correction.

### DESCRIPTION OF RELATED ART

The literature has put forth several formulations for sunscreen compositions. These include:

Published Patent Application Number US 2012/0128606 which describes a variety of skin products including a Skin Beautifying Milk, Body Creme, Fade Creme and MaxiTone;
Published Patent Application Number US 2009/0169608 which describes a topically applicable composition for use as a skin bleaching agent;
U.S. Pat. No. 7,842,723 which describes ascorbic acid—natural sugar lactone esters for comprehensive skin and scalp care;
Published Patent Application Number US 2011/0229536 which describes compositions for topical applications comprising microencapsulated colorants;
Published Patent Application Number US 2012/0157939 which describes compositions comprising lilium siberia extracts and uses thereof;
Published Patent Application Number US 2004/0166069 which describes boosting tyrosinase inhibiting activity of skin whitening and sunscreen compositions;
U.S. Pat. No. 6,024,942 which describes photoprotective compositions;
Published PCT Application WO 2004/043422 which describes an improved cosmetic composition;
Published U.S. Patent Application Number US 2011/0206793 which describes topical skin care formulation;

None of these references describe the present invention.

### SUMMARY OF THE INVENTION

It is an object of the present invention to provide a sunscreen composition that also provides pigment correction. The instant composition can be used as a face or a body cream. In addition, the composition can be used to treat various skin pigment conditions including lentigo, liver spots, melasma, post-inflammatory pigment issues, sunspots, freckles, and age spots.

In a preferred embodiment, the sunscreen composition comprises zinc oxide; octinoxate; α-arbutin; ascorbic acid; glycyrrhiza glabra extract; green tea extract; pomegranate extract; cucumber extract; niacinamide; and a carrier.

In the most preferred embodiment, the sunscreen composition comprises zinc oxide; octinoxate; α-arbutin; aloe barbadensis leaf juice; water; ascorbic acid; bis-vinyl dimethicone copolymer; camellia sinensis leaf (green tea) extract; C13-14 isoparaffin; caprylic/capric triglyceride; cucumis sativus fruit extract; cyclomethicone; dimethicone; ethylhexylglycerin; glycerin; glyceryl stearate; glycyrrhiza glabra extract; helianthus annuus oil; laureth-7; niacinamide; parfum; PEG-100 stearate; phenoxyethanol; polyacrylamide; polysorbate-20; punica granatum (pomengranate) extract; and tocopheryl acetate.

The present invention also includes a method of protecting the skin against the sun and correcting a pigment issue comprising applying a sunscreen composition to the skin of a person in need of sunscreen and pigment correction, said sunscreen composition comprising zinc oxide; octinoxate; α-arbutin; ascorbic acid; glycrrhiza glabra extract; green tea extract; pomegranate extract; cucumber extract; niacinamide; and a carrier.

The pigment issues that can be corrected include liver spots, melasma and post-inflammatory pigment issues.

In its most preferred embodiment, the method of protecting the skin against the sun and correcting a pigment issue comprises applying a sunscreen composition to the skin of a person in need of sunscreen and pigment correction, said sunscreen composition comprising: zinc oxide; octinoxate; α-arbutin; aloe barbadensis leaf juice; water; ascorbic acid; bis-vinyl dimethicone copolymer; camellia sinensis leaf (green tea) extract; C13-14 isoparaffin; caprylic/capric triglyceride; cucumis sativus fruit extract; cyclomethicone; dimethicone; ethylhexylglycerin; glycerin; glyceryl stearate; glycyrrhiza glabra extract; helianthus annuus oil; laureth-7; niacinamide; parfum; PEG-100 stearate; phenoxyethanol; polyacrylamide; polysorbate-20; punica granatum (pomegranate) extract; and tocopheryl acetate.

### DETAILED DESCRIPTION OF THE INVENTION

Any suitable carrier may be used in the compositions of the present invention. Preferably, for a skin care composition, the carrier is a cosmetically-acceptable carrier. As will be recognized by those of skill in the art, cosmetically acceptable carriers comprise carriers that are suitable for use in contact with the body, in particular the skin for pigment correcting application, without undue toxicity, incompatibility, instability, irritation, allergic response, and the like.

The carrier can be in a wide variety of forms. For example, emulsion carriers, including, but not limited to, oil-in-water, water-in-oil, water-in-oil-in-water, and oil-in-water-in-silicone emulsions. The emulsions can cover a wide range of viscosities.

Examples of suitable cosmetically-acceptable carriers include cosmetically-acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, make-up, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like. The formulation of an acceptable carrier is within the skill of those in the art.

In a preferred embodiment, the composition has a SPF rating of 15. The SPF rating can be adjusted.

Although the invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

What is claimed is:

1. A sunscreen composition comprising:
zinc oxide;
octinoxate;
α-arbutin;
ascorbic acid;
glycyrrhiza glabra extract;
green tea extract;
pomegranate extract;
cucumber extract;
niacinamide; and
a carrier.

2. A sunscreen composition comprising:
zinc oxide;
octinoxate;
α-arbutin;
aloe barbadensis leaf juice;
water;
ascorbic acid;
bis-vinyl dimethicone copolymer;
camellia sinensis leaf extract;
C13-14 isoparaffin;
caprylic/capric triglyceride;
cucumis sativus fruit extract;
cyclomethicone;
dimethicone;
ethylhexylglycerin;
glycerin;
glyceryl stearate;
glycrrhiza glabra extract;
helianthus annuus oil;
laureth-7;
niacinamide;
parfum;
PEG-100 stearate;
phenoxyethanol;
polyacrylamide;
polysorbate-20;
punica granatum extract; and
tocopheryl acetate.

3. A method of protecting the skin against the sun and a correcting pigment issue comprising
applying a sunscreen composition to the skin of a person in need of sunscreen and pigment correction,
said sunscreen composition comprising:
zinc oxide;
octinoxate;
α-arbutin;
ascorbic acid;
glycyrrhiza glabra extract;
green tea extract;
pomegranate extract
cucumber extract;
niacinamide; and
a carrier.

4. The method of claim 3, wherein the pigment issue is a liver spot.

5. The method of claim 3, wherein the pigment issue is melasma.

6. The method of claim 3, wherein the pigment issue is a post-inflammatory pigment issue.

7. A method of protecting the skin against the sun and a correcting pigment issue comprising
applying a sunscreen composition to the skin of a person in need of sunscreen and pigment correction,
said sunscreen composition comprising:
zinc oxide;
octinoxate;
α-Arbutin;
aloe barbadensis leaf juice;
water;
ascorbic acid;
bis-vinyl dimethicone copolymer;
camellia sinensis leaf extract;
C13-14 isoparaffin;
caprylic/capric triglyceride;
cucumis sativus fruit extract;
cyclomethicone;
dimethicone;
ethylhexylglycerin;
glycerin;
glyceryl stearate;
glycrrhiza glabra extract;
helianthus annuus oil;
laureth-7;
niacinamide;
parfum;
PEG-100 stearate;
phenoxyethanol;
polyacrylamide;
polysorbate-20;
punica granatum extract; and
tocopheryl acetate.

8. The method of claim 7, wherein the pigment issue is a liver spot.

9. The method of claim 7, wherein the pigment issue is melasma.

10. The method of claim 7, wherein the pigment issue is a post-inflammatory pigment issue.
